(19) United States
(12) Patent Application Publication
(10) Pub. No.: US 2009/0275053 A1
(43) Pub. Date: Nov. 5, 2009

(54) CELL-BASED PCSK9 SCREENING ASSAY

(75) Inventors: Jay D. Horton, Plano, TX (US); Markey C. McNutt, Dallas, TX (US)

Correspondence Address: CHALKER FLORES, LLP, 2711 LBJ FRWY, Suite 1036, DALLAS, TX, 75234, US

(73) Assignee: Board of Regents, The University of Texas System, Austin, TX (US)

(21) Appl. No.: 12/112,615

(22) Filed: Apr. 30, 2008

(51) Int. Cl. G01N 33/53

(52) U.S. Cl. 435/7.6

(57) ABSTRACT

The present invention includes a PCSK9 activity inhibition assay system, kits, compositions and methods. The present invention includes a cell having a first vector capable of expressing a catalytic fragment of PCSK9, a second vector capable of expressing a prodomain of PCSK9 and a V5 protein with a detectable label. The V5 protein forms a fusion protein with the prodomain of PCSK9 and wherein cleavage of the prodomain by the catalytic fragment of PCSK9 releases a detectable signal.

### STATEMENT OF FEDERALLY FUNDED RESEARCH

This invention was made with U.S. Government support under Contract No. HL 20948 awarded by the NIH. The government has certain rights in this invention.

### TECHNICAL FIELD OF THE INVENTION

The present invention relates in general to the field of cell based screening and more specifically to screening assays for serine proteases.

### BACKGROUND OF THE INVENTION

Without limiting the scope of the invention, its background is described in connection with cell based screening assays and more specifically to screening assays for regulators of serine protease activity and regulators of serum cholesterol levels.

Proprotein convertase subtilisin/kexin type 9 (PCSK9) is a gene that encodes a proprotein convertase belonging to the proteinase K subfamily of the secretory subtilase family. The encoded protein is synthesized as a soluble zymogen that undergoes autocatalytic intramolecular processing and plays a crucial role in cholesterol homeostasis . Mutations in this gene that cause increased activity have been associated with a form of autosomal dominant familial hypercholesterolemia (HCHOLA3). Conversely, mutations in the PCSK9 that result in reduced activity result in very low blood cholesterol levels and those indiviuals carrying these mutations have an 88% reduction in cardiovascular events. The Proprotein convertase subtilisin/kexin type 9 gene can also be referred to as HCHOLA3, NARC1, neural apoptosis regulated convertase 1, proprotein convertase PC9, or subtilisin/kexin-like protease PC9.

Generally, the PCSK9 plays a role in regulating blood cholesterol levels through the altering the number of receptors that bindof low-density lipoproteins. The number of low-density lipoprotein receptors on the surface of liver cells determines how quickly cholesterol is removed from the bloodstream. The PCSK9 protein is secreted into the blood and controls blood cholesterol levels by breaking down low-density lipoprotein receptors.

Modifications of the PCSK9 gene that increase the activity of the protein are associated with an inherited form of high cholesterol (hypercholesterolemia). The overactive PCSK9 protein significantly reduces the number of low-density lipoprotein receptors on the surface of liver cells. Low-density lipoprotein receptors are responsible for removing LDL cholesterol from the blood.The overactive PCSK9 protein causes very high blood cholesterol levels, which may be deposited abnormally in tissues such as the skin, tendons, and arteries that supply blood to the heart (coronary arteries). A buildup of cholesterol in the walls of coronary arteries greatly increases the risk of a heart attack.

U.S. Pat. No. 7,300,754 entitled, “Methods for detecting the presence of or predisposition to autosomal dominant hypercholesterolemia” discloses the identification of a human hypercholesterolemia causal gene, which can be used for the diagnosis, prevention and treatment of hypercholesterolemia, more particularly familial hypercholesterolemia, as well as for the screening of therapeutically active drugs. The patent more specifically disclosed that mutations in the PCSK9 gene encoding NARC-1 causes autosomal dominant hypercholesterolemia and represent novel targets for therapeutic intervention that can be used in the diagnosis of predisposition to, detection, prevention and/or treatment of coronary heart disease and, cholesterol, lipid and lipoprotein metabolism disorders, including familial hypercholesterolemia, atherogenic dyslipidemia, atherosclerosis, cardiovascular diseases.

### SUMMARY OF THE INVENTION

The present inventors recognized that the expression of LDL receptors is the primary mechanism by which humans lower LDL cholesterol in the blood and that by modifying the PCSK9 protein results in an increase in LDL receptors and a significant lowering of LDL cholesterol. It was also recognized that there were no known small molecule inhibitors of PCSK9 and there was no in vitro activity assays for PCSK9. All of the assays used in the art have a high false positive rate that results from non-specific molecules that generally inhibit protein secretion from the cell, e.g., compounds that affects viability, transcription, translation, or cell trafficking.

The present invention provides compositions and methods for a PCSK9 activity inhibition assay system. The assay system includes a cell having a first vector capable of expressing a catalytic fragment of PCSK9, a second vector capable of expressing a prodomain of PCSK9 and a V5 protein as a detectable label. The V5 protein forms a fusion protein with the prodomain of PCSK9 and wherein cleavage of the prodomain by the catalytic fragment of PCSK9 releases a detectable signal.

The present invention also includes a cell-based method of screening molecules for inhibition of PCSK9 catalytic activity by providing one or more cells having a first vector capable of expressing a catalytic fragment of PCSK9, a second vector capable of expressing a prodomain of PCSK9 and a source of V5 protein having a detectable label to form a V5 prodomain chimera with the V5 protein linked to the C-terminus of the prodomain. The cells are incubated with a sample suspected of having a PCSK9 catalytic activity inhibitor and the presence of the detectable label is detected. The catalytic fragment of PCSK9 cleaves the detectable label to provide a cell-based assay that can be used to screen molecules for inhibition of PCSK9 catalytic activity.

The present invention provides a PCSK9 inhibition assay kit. The kit includes a first vector capable of expressing a catalytically active fragment of PCSK9, a second vector capable of expressing a prodomain of PCSK9 and a detectable label. The detectable label forms a detectable label-prodomain fusion protein and the catalytic fragment of PCSK9 cleaves the detectable label to provide a cell-based assay to screen molecules for inhibition of PCSK9 catalytic activity.

The present invention provides an inhibition assay including one or more cells with a first expression vector capable of expressing a catalytic protein fragment, a second expression vector capable of expressing a prodomain fragment and an indicator cleavably linked to the prodomain fragment. The catalytic fragment cleaves the indicator from the prodomain fragment to provide a detectable indicator that can be used to screen molecules for inhibition of the activity of the catalytic protein fragment. The present invention provides an inhibition assay having a catalytic protein fragment disposed within a cell and a prodomain fragment comprising an indicator disposed within the cell. The catalytic fragment cleaves the indicator from the prodomain fragment to indicate catalytic activity.

The present invention also includes a method of determining PCSK9 activity inhibition by providing an inhibition assay comprising one or more HEK-293 cells having a catalytic fragment expression vector capable of expressing amino acids 153-692 of a catalytic fragment of PCSK9, a prodomain expression vector capable of expressing amino acids 31-152 of a prodomain of PCSK9 and a GFP labeled V5 protein capable of forming a V5-prodomain of PCSK9 fusion protein. The catalytic fragment of PCSK9 cleavage of the GFP from the V5-prodomain of PCSK9 fusion protein to releases the GFP as a detectable label. The inhibition assay is contacted with a sample suspected of comprising a PCSK9 activity inhibitor and the presence of the GFP label is detected to determine the inhibition of the PCSK9 activity inhibitor.

The present invention also includes a PCSK9 activity inhibition assay system that includes one or more HEK-293 cells. The cells include a catalytic fragment expression vector capable of expressing amino acids 153-692 of a catalytic fragment of PCSK9, a prodomain expression vector capable of expressing amino acids 31-152 of a prodomain of PCSK9 and a GFP labeled V5 protein capable of forming a V5-prodomain of PCSK9 fusion protein. The catalytic fragment of PCSK9 cleavage of the GFP from the V5-prodomain of PCSK9 fusion protein to releases the GFP as a detectable label.

### BRIEF DESCRIPTION OF THE DRAWINGS

For a more complete understanding of the features and advantages of the present invention, reference is now made to the detailed description of the invention along with the accompanying figures and in which:

FIG. 1 is an image of a gel showing the activity of the catalytic fragment of PCSK9;

FIG. 2 is a schematic diagram of PCSK9 expressed as two peptides with GFP fused to the prodomain;

FIG. 3 is a graph of the stability of transfected HEK-293 cell lines expressing PCSK9-GFP and PCSK9 S386A; and

FIG. 4 is a graph of a sample assay.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term PCSK9 is used interchangeably with proprotein convertase subtilisin/kexin type 9, HCHOLA3, NARC1, neural apoptosis regulated convertase 1, proprotein convertase PC9, and/or subtilisin/kexin-like protease PC9.

These vectors can be used to express a polypeptide according to the present invention in vitro, ex vivo or in vivo, to create transgenic or “Knock Out” non-human animals, to amplify the nucleic acids, to express antisense RNAs, etc.

As used herein, the term vector may be a plasmid, a virus, a cosmid, a phage, a BAC, a YAC, etc. Plasmid vectors may be prepared from commercially available vectors such as pBluescript, pUC, pBR, etc. Viral vectors may be produced from baculoviruses, retroviruses, adenoviruses, AAVs, etc., according to recombinant DNA techniques known in the art.

The vectors of this invention typically include a coding sequence operably linked to regulatory sequences, e.g., a promoter, a polyA, etc. The term “operably linked” indicates that the coding and regulatory sequences are functionally associated so that the regulatory sequences cause expression (e.g., transcription) of the coding sequences. The vectors may further comprise one or several origins of replication and/or selectable markers. The promoter region may be homologous or heterologous with respect to the coding sequence, and provide for ubiquitous, constitutive, regulated and/or tissue specific expression, in any appropriate host cell, including for in vivo use. Examples of promoters include bacterial promoters (T7, pTAC, Trp promoter, etc.), viral promoters (LTR, TK, CMV-IE, etc.), mammalian gene promoters (albumin, PGK, etc), and the like.

As used herein, the term “diagnosis” includes the detection, monitoring, dosing, comparison, etc., at various stages, including early, pre-symptomatic stages, and late stages, in adults, children and pre-birth. Diagnosis typically includes the prognosis, the assessment of a predisposition or risk of development, the characterization of a subject to define most appropriate treatment (pharmaco-genetics), etc.

As used herein, the terms “markers,” “detectable markers” and “detectable labels” are used interchangeably to refer to compounds and/or elements that can be detected due to their specific functional properties and/or chemical characteristics, the use of which allows the agent to which they are attached to be detected, and/or further quantified if desired, such as, e.g., an enzyme, radioisotope, electron dense particles, magnetic particles or chromophore. There are many types of detectable labels, including fluorescent labels, which are easily handled, inexpensive and nontoxic. Detectable labels may also be proteins or combinations of proteins, e.g., antibodies that carry a detectable agent, enzyme, or chromophores. Green fluorescent protein or “GFP” is one example of a detectable label that has gained widespread use as a tool to visualize spatial and temporal patterns of gene expression in vivo that may be used with the present invention.

The serine protease PCSK9 is a major determinant serum cholesterol levels in humans. Individuals who are heterozygous for inactivation mutations of the PCSK9 gene have on average a 28% reduction in plasma LDL cholesterol and an 88% reduction in risk for coronary heart disease (1,2). Inhibition of PCSK9 function presents a novel therapeutic target for the treatment of hypercholesterolemia. Proteolytic activity of PCSK9 is required for proper maturation and secretion of the protein; however, catalytic activity is not directly involved in its regulation of plasma cholesterol levels (3)(4). As a result, small-molecule inhibitors of PCSK9 catalytic activity must be cell permeable to block PCSK9 action.

The present inventors recognized that there were no known small molecule inhibitors of PCSK9 and no in vitro activity assays for PCSK9. PCSK9 undergoes intramolecular self-cleavage but it has no other known proteolytic substrates. As a result, cell-based assays that measure PCSK9 secretion have been used as a surrogate for PCSK9 activity. The most common method being the use of a reporter such as luciferase fused to PCSK9. If PCSK9 is active, it undergoes normal secretion and reporter activity and can be detected in the medium. If activity is blocked, PCSK9 fails to be secreted and reporter activity goes down. The major limitation of these assays is a high false positive rate that results from non-specific molecules that generally inhibit protein secretion from the cell. Generally, any compound that affects viability, transcription, translation, or cell trafficking will result in a decrease in reporter signal.

The present inventors developed a secretion assay that overcomes these problems and limitations. The present inventors recognized that PCSK9 could be expressed as two separate peptide chains, a prodomain fragment and a catalytic fragment, which associate resulting in an active secreted protein (3). These two fragments reflect the cleavage location of full-length PCSK9, thereby eliminating the need for catalytic activity to produce a mature protein. The present inventors also recognized that a V5 tag placed at the C-terminus of the prodomain fragment was cleaved off of the prodomain once the mature protein formed. Importantly, the V5 tag was not cleaved if an inactivating mutation was made in the catalytic fragment.

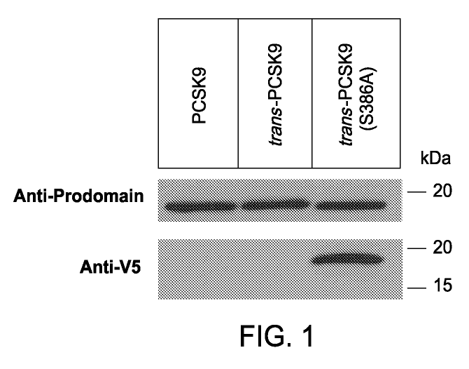

FIG. 1 is a gel showing the activity of the catalytic fragment of PCSK9. The gel image in FIG. 1 shows PCSK9 was expressed in full-length form (lane 1) or as two separate peptides (trans-PCSK9) (lanes 2 and 3). Medium was immunoblotted for PCSK9 prodomain or V5 tag. Full-length catalytically active does not contain a V5 tag and serves as a negative control. Catalytically active PCSK9 expressed as two peptides effectively cleaves a V5 tag from the prodomain, thus the anti-V5 antibody does not produce a signal in the medium. In the absence of catalytic activity (lane 3) the V5-tag remains associated with the prodomain and is detected in the medium. This shows that the tag recapitulates the natural PCSK9 cleavage site. The present inventors determined that a GFP reporter could also be cleaved from the prodomain.

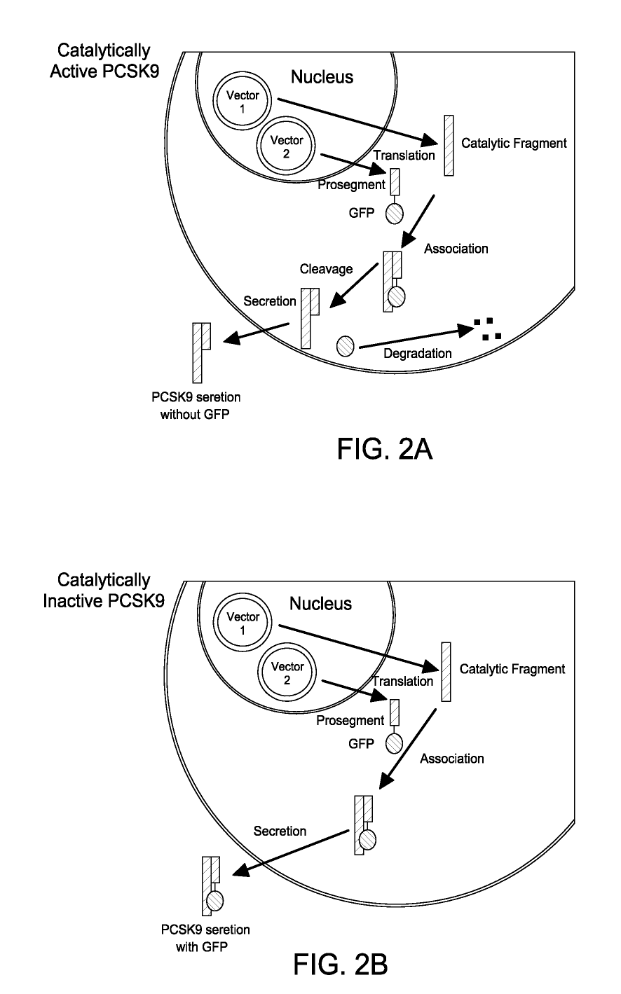

FIGS. 2A and 2B are schematic diagrams of PCSK9 expressed as two peptides with GFP fused to the prodomain. When PCSK9 is catalytically active (FIG. 2A) GFP is cleaved in the cell and PCSK9 is secreted without GFP. When PCSK9 is inactive (FIG. 2B), GFP is secreted still linked to PCSK9. The ability of the PCSK9 to cleave the GFP reporter provides a cell-based assay that can be used to screen small molecules for inhibition of PCSK9 catalytic activity. In one embodiment the assay uses HEK-293 cells that are stably transfected with two plasmids. The first plasmid expresses the catalytic fragment of human PCSK9 (amino acids 153-692) and the second plasmid expresses the prodomain of human PCSK9 (amino acids 31-152) with a V5-GFP linked to the C-terminus. These cells can be used in a cell-based screen for PCSK9 inhibitors.

FIG. 2A shows the basal state GFP is cleaved from PCSK9. FIG. 2A is a schematic of the catalytic activity PCSK9 10 assay. The cell 12 has a nucleus 14 that contains a first vector 16 and a second vector 18. The first vector 16 and second vector 18 undergo translation 20 to form a catalytic fragment 22 and a prosegment 24 attached to GFP 26. The catalytic fragment 22 and prosegment 24 and GFP 26 associate 28 into a complex 30. The complex 30 then undergoes cleavage 32 to form secretion 34 and GFP 26. The GFP 26 is degraded 36 into components 38 that remain within the cell 12. Secretion 34 is secreted from the cell 12 to form a PCSK9 Secretion without GFP 40.

When a compound blocks PCSK9 catalytic activity as in FIG. 2B, GFP cleavage is prevented and a PCSK9-GFP fusion protein will be secreted intact into the medium. Accumulation of PCSK9-GFP in the medium is a direct readout of inhibition of PCSK9 activity. FIG. 2B shows the basal state GFP is not cleaved from PCSK9. FIG. 2B is a schematic of the catalytic inactivity PCSK9 42 assay. The cell 12 has a nucleus 14 that contains a first vector 16 and a second vector 18. The first vector 16 and second vector 18 undergo translation 20 to form a catalytic fragment 22 and a prosegment 24 attached to GFP 26. The catalytic fragment 22 and prosegment 24 and GFP 26 associate 28 into a complex 30. The complex 30 does not undergoe cleavage (not shown). Complex 30 is secreted from the cell 12 to form a PCSK9 Secretion with GFP 44.

The presence of PCSK9-GFP in the medium is detected using a sandwich ELISA assay. Anti-GFP antibody is used to capture proteins and our anti-PCSK9 antibody is used for detection. Our PCSK9 antibody is a monoclonal antibody that detects an epitope in the catalytic domain of PCSK9. Using this combination of antibodies, the captured epitope is on the prodomain-GFP fusion peptide and the detection epitope is on the catalytic fragment of PCSK9 ensuring that free GFP, PCSK9 without the GFP fusion, and dissociated PCSK9 prodomain and catalytic domain are not detected. Quantification of the ELISA is performed by using a peroxidase linked secondary antibody and a chemiluminescent substrate and reading luminescence.

The assay cell line secretes only PCSK9 that has GFP cleaved from the prodomain and results in only a background signal being detected by the ELISA. If a compound inactivates PCSK9, PCSK9-GFP fusion protein will accumulate in the medium and the ELISA values will become non-zero; if a compound has no effect on PCSK9, ELISA values will remain at background. Inasmuch as this assay looks for an appearance of a signal, compounds that affect the secretion of PCSK9 or the viability of cells will not show up as false positives as they do in other secretion assays.

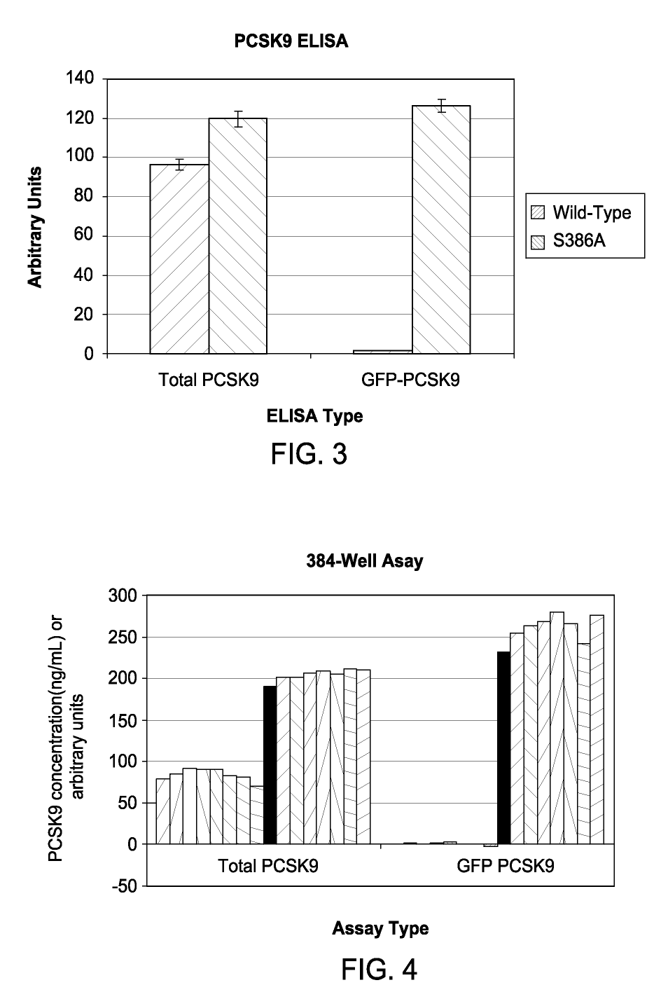

FIG. 3 is a graph of the stability of transfected HEK-293 cell lines expressing catalytically trans-PCSK9-GFP and trans-PCSK9 S386A. The graph shows data from the ELISA of stably transfected HEK-293 cell lines expressing catalytically active trans-PCSK9-GFP (wild-type) and catalytically inactive trans-PCSK9 S386A). The secretion of PCSK9 from the cells is similar based on total PCSK9 protein quantified by an ELISA; however, the wild-type type cell line effectively cleaves all the GFP from the secreted PCSK9 and no GFP-PCSK9 is detected in the medium. An inhibitor of PCSK9 catalytic activity should increase the amount of GFP-PCSK9 in the medium of the wild-type cell line.

To determine the ELISA values expected from an inhibitory compound, a positive control must be used. Since there are no known compounds that inhibit PCSK9 catalytic activity, a second stable cell line was created as a control. This control cell line expresses PCSK9 as two peptides as described for the assay cell line, but contains an inactivating mutation (S386A) in the catalytic fragment of PCSK9. These cells only secrete a PCSK9-GFP fusion protein. In the graph of FIG. 3 the control cells produce a clear signal in the ELISA, which represents the maximum signal that will be seen if a compound is 100% effective at blocking PCSK9 catalytic activity. Using the signal values from the control cell line as a reference, a threshold luminescence value, e.g. 10% of control value, can be set as a cutoff for determining if a compound is a “hit” in the assay.

FIG. 4 is a graph of a sample assay showing sample assay data from 8 replicate wells for each cell line. The first 8 bars represent the wild-type cell line (WT) and the last 8 represent the catalytically inactive cell line (S386A). As seen in FIG. 1, both cell lines secrete PCSK9; however the wild-type cell line cleaves all GFP from the PCSK9 fusion molecule and is not detected in the GFP ELISA. Additionally, these data show the low variance between wells and the reproducibility of the assay. Total PCSK9 is measured in ng/mL. GFP PCSK9 is measured in arbitrary units.

Plasmids. A plasmid containing the prodomain of PCSK9 (amino acids 1-152) followed by a C-terminal V5 tag and green fluorescent protein (GFP) protein from Aequorea victoria (5) was created. The PCSK9 prodomain and V5 sequence was amplified by PCR as described (3). The PCR fragment was ligated into the pcDNA3.1/CT-GFP-TOPO vector (Invitrogen) using the manufacturer's instructions. The resulting plasmid was mutagenized to move the GFP in frame directly after the V5 tag using the QuikChange site-directed mutagenesis kit (Statagene) and the primer SEQ. ID No.: 1: 5′-CTCCTCGGTCTCGATTCTACGATGGCTAGCAAAGGAGAAGAAC-3′. The prodomain-V5-GFP sequence was transferred to the pcDNA3.1/Hyrgo(+) vector (Invitrogen) through restriction digestion with KPNI and XBAI. Plasmids containing a deletion of the prodomain of PCSK9 with or without a mutation at serine 386 to alanine were previously generated (3).

Cell Lines. Human embryonic kidney (HEK) 293 cells (CRL-1573) were stably co-transfected with PCSK9 prodomain-V5-GFP and PCSK9 catalytic and terminal portions with either wild-type sequence or the catalytically inactivating mutation S386A. Cells were cultures in DMEM (cellgro; Mediatech, Inc.) supplemented with 100 U/mL penicillin, 100 μg/mL streptomycin, 1 g/L glucose, and 10% FCS. Cells were plated at 5×10⁶ cells per 100mm dish on day 0. On day 3, the medium was replaced and the cells co-transfected with 1.5 ug prodomain plasmid and 0.5 μg catalytic and C-terminus plasmid per dish using Lipofectamine 2000 transfection reagent (Invitrogen). On day 4, cells were switched to a selection media containing 500 μg/mL Hygromycin and 700 μg/mL G418. Surviving colonies were selected and screened for secretion of PCSK9 using western blot analysis. The colonies with highest secretion of wild-type and S386A PCSK9 were sub-cloned from single-cells and re-screened for highest PCSK9 expression.

Screening Assay. The cell lines with stable expression of either wild-type or S386A PCSK9 are plated into 384 well plates (Corning, #3701) at densities of 105⁵ in 80 μL of the medium described above. The next day the compound to be screened or vehicle can be added directly into the wells. After 18 hours incubation, 40 μL of medium are removed to each of 2 assay plates. One plate is a MaxiSorp 384 well plate (NUNC, #460372) pre-coated with the polyclonal anti-PCSK9 antibody 228B and the other plate is a ChoiceCoat Custom-coated anti-GFP plate (Pierce). Detection of captured PCSK9 in both plates is carried out with a monoclonal PCSK9 antibody as described (6).

The present invention provides a PCSK9 activity inhibition assay system. The system includes a cell having a first vector capable of expressing a catalytic fragment of PCSK9, a second vector capable of expressing a prodomain of PCSK9 and a V5 protein with a detectable label. The V5 protein forms a fusion protein with the prodomain of PCSK9 and wherein cleavage of the prodomain by the catalytic fragment of PCSK9 releases a detectable signal. In some embodiments of the present invention the first vector, the second vector or both the first vector and the second vector comprises an expression vector and the catalytic fragment of PCSK9 may include all or a portion of the amino acids 153-692 of PCSK9 and the prodomain of PCSK9 may include all or a portion of the amino acids 31-152 of PCSK9. Although GFP is used as a detectable label, the skilled artisan will know other detectable label may be used. Similarly, the present invention may use HEK-293 cells but other cell lines may be used.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Kotowski, I. K., Pertsemlidis, A., Luke, A., Cooper, R. S., Vega, G. L., Cohen, J. C., and Hobbs, H. H. (2006) Am J Hum Genet 78(3), 410-422

2. Cohen, J. C., Boerwinkle, E., Mosley, T. H., Jr., and Hobbs, H. H. (2006) N Engl J Med 354(12), 1264-1272

3. McNutt, M. C., Lagace, T. A., and Horton, J. D. (2007) J Biol Chem 282(29), 20799-20803

4. Park, S. W., Moon, Y. A., and Horton, J. D. (2004) J Biol Chem 279(48), 50630-50638

5. Crameri, A., Whitehorn, E. A., Tate, E., and Stemmer, W. P. (1996) Nature biotechnology 14(3), 315-319

6. Lagace, T. A., Curtis, D. E., Garuti, R., McNutt, M. C., Park, S. W., Prather, H. B., Anderson, N. N., Ho, Y. K., Hammer, R. E., and Horton, J. D. (2006) The J. of Clin. Invest. 116(11), 2995-3005

What is claimed is:

1. A PCSK9 activity inhibition assay system comprising:
a cell comprising:
a first vector capable of expressing a catalytic fragment of PCSK9;
a second vector capable of expressing a prodomain of PCSK9; and
a V5 protein comprising a detectable label, wherein the V5 protein forms a fusion protein with the prodomain of PCSK9 and wherein cleavage of the prodomain by the catalytic fragment of PCSK9 releases a detectable signal.

2. The assay system of claim 1, wherein the first vector, the second vector or both the first vector and the second vector comprises an expression vector.

3. The assay system of claim 1, wherein the cells comprise HEK-293 cells.

4. The assay system of claim 1, wherein the catalytic fragment of PCSK9 comprises amino acids 153-692 of PCSK9 and the prodomain of PCSK9 comprises amino acids 31-152 of PCSK9.

5. The assay system of claim 1, wherein the detectable label comprises GFP.

6. A cell-based method of screening molecules for inhibition of PCSK9 catalytic activity comprising the steps of:
providing one or more cells comprising a first vector capable of expressing a catalytic fragment of PCSK9, a second vector capable of expressing a prodomain of PCSK9 and a source of V5 protein having a detectable label to form a V5 prodomain chimera, wherein the V5 protein is linked to the C-terminus of the prodomain;
contacting the one or more cells with a sample suspected of comprising a PCSK9 catalytic activity inhibitor; and
detecting the presence of the detectable label, wherein the catalytic fragment of PCSK9 cleaves the detectable labelto provide a cell-based assay that can be used to screen molecules for inhibition of PCSK9 catalytic activity.

7. The method of claim 6, wherein the vector comprises an expression vector.

8. The method of claim 6, wherein the one or more cells comprises HEK-293 cells.

9. The method of claim 6, wherein the catalytic fragment of PCSK9 comprises amino acids 153-692 of PCSK9 and the prodomain of PCSK9 comprises amino acids 31-152 of PCSK9.

10. The method of claim 6, wherein the detectable protein comprises GFP.

11. A PCSK9 inhibition assay kit comprising:
a first vector capable of expressing a catalytically active fragment of PCSK9;
a second vector capable of expressing a prodomain of PCSK9; and
a detectable label to form a detectable label prodomain fusion protein, wherein the catalytic fragment of PCSK9 cleaves the detectable label to provides a cell-based assay to screen molecules for inhibition of PCSK9 catalytic activity.

12. The kit of claim 11, further comprising one or more HEK-293 transfection cells.

13. The kit of claim 11, wherein the vector comprises an expression vector.

14. The kit of claim 11, wherein the catalytic fragment of PCSK9 comprises amino acids 153-692 of PCSK9 and the prodomain of PCSK9 comprises amino acids 31-152 of PCSK9.

15. An inhibition assay comprising:
one or more cells comprising a first expression vector capable of expressing a catalytic protein fragment,
a second expression vector capable of expressing a prodomain fragment and
an indicator cleavably linked to the prodomain fragment, wherein the catalytic fragment cleaves the indicator from the prodomain fragment to provide a detectable indicator that can be used to screen molecules for inhibition of the activity of the catalytic protein fragment.

16. The assay of claim 15, wherein the vector comprises a plasmid and the one or more cells

17. The assay of claim 15, wherein the catalytic protein fragment comprises amino acids 153-692 of PCSK9 and the prodomain fragment comprises amino acids 31-152 of PCSK9.

18. An inhibition assay comprising:
a catalytic protein fragment disposed within a cell, and
a prodomain fragment comprising an indicator disposed within the cell, wherein the catalytic fragment cleaves the indicator from the prodomain fragment to indicate catalytic activity.

19. A method of determining PCSK9 activity inhibition comprising the steps of:
providing an inhibition assay comprising one or more HEK-293 cells comprising a catalytic fragment expression vector capable of expressing amino acids 153-692 of a catalytic fragment of PCSK9, a prodomain expression vector capable of expressing amino acids 31-152 of a prodomain of PCSK9 and a GFP labeled V5 protein capable of forming a V5-prodomain of PCSK9 fusion protein, wherein the catalytic fragment of PCSK9 cleavage of the GFP from the V5-prodomain of PCSK9 fusion protein to releases the GFP as a detectable label
contacting the inhibition assay with a sample suspected of comprising a PCSK9 activity inhibitor; and
detecting the presence of the GFP label to determine the inhibition of the PCSK9 activity inhibitor.

20. A PCSK9 activity inhibition assay system comprising:
one or more HEK-293 cells comprising
a catalytic fragment expression vector capable of expressing amino acids 153-692 of a catalytic fragment of PCSK9;
a prodomain expression vector capable of expressing amino acids 31-152 of a prodomain of PCSK9; and
a GFP labeled V5 protein capable of forming a V5-prodomain of PCSK9 fusion protein, wherein the catalytic fragment of PCSK9 cleavage of the GFP from the V5-prodomain of PCSK9 fusion protein to releases the GFP as a detectable label.


SEQUENCE LISTING (1 sequence)

SEQ ID NO: 1 - DNA, 43 bp, Artificial Sequence
  Synthetic oligonucleotide
ctcctcggtctcgattctacgatggctagcaaaggagaagaac